(19) United States
(12) Patent Application Publication
(10) Pub. No.: US 2023/0285134 A1
(43) Pub. Date: Sep. 14, 2023

(54) Embolic Protection System

(71) Applicant: The Medical College of Wisconsin, Inc., Milwaukee, WI (US)

(72) Inventors: David Joyce, Milwaukee, WI (US); Lyle Joyce, Milwaukee, WI (US); Michael Salinger, Milwaukee, WI (US); Brian Lynch, Milwaukee, WI (US)

(21) Appl. No.: 18/040,459

(22) PCT Filed: Aug. 3, 2021
(86) PCT No.: PCT/US2021/044364

§ 371 Date: Feb. 3, 2023

Related U.S. Application Data
(60) Provisional application No. 63/060,588, filed on Aug. 3, 2020.

(51) Int. Cl. A61F 2/01; A61M 60/178

(52) U.S. Cl. CPC A61F 2/014; A61F 2/011; A61F 2/0105; A61M 60/178; A61F 2210/0014; A61M 2025/1052

(57) ABSTRACT

An embolic protection device includes an expandable mesh net coupled to a catheter assembly used in an interventional procedure. The catheter assembly includes an outer catheter, an inner catheter, a compliant balloon, and the mesh net. The catheter assembly can be deployed in a lumen, such as an outflow graft of a left ventricular assist device, a blood vessel, or other bodily lumen. The mesh net is coupled to the inner catheter and is expanded by retracting the outer catheter when positioned in the lumen. The mesh net captures embolic debris before it is able to enter into the patient's blood stream.

### CROSS-REFERENCE TO RELATED APPLICATIONS

This application claims the benefit of U.S. Provisional Patent Application Ser. No. 63/060,588, filed on Aug. 3, 2020, and entitled “EMBOLIC PROTECTION SYSTEM,” which is herein incorporated by reference in its entirety.

### BACKGROUND

Heart failure affects 5.8 million Americans, with an expanding prevalence as over 670,000 new cases are diagnosed each year. Mechanical Circulatory Support (“MCS”) has been shown to dramatically improve survival and quality of life among patients with end-stage heart failure. However, thrombotic complications can be prevalent in MCS systems if not addressed. Pump thrombosis can be treated by exchanging the MCS; however, current techniques for device exchange require either a large subcostal incision with cannulation via femoral cutdown or a redo-sternotomy approach. These procedures are highly invasive, requiring cardiopulmonary bypass and are associated with considerable morbidity and the potential for mortality.

Lytic therapy represents the only currently available alternative, which can result in cerebrovascular accident. Many patients pose the additional risk of requiring multiple device exchanges for recurrent thrombosis episodes. Even in an uncomplicated device exchange, hospitalization is typically required for a week or more.

Thus, it would be advantageous to provide a system for treating pump thrombosis in patients with MCS that does not require total MCS replacement, and also protects against potential damage by emboli formation.

### SUMMARY OF THE DISCLOSURE

It is an aspect of the present disclosure to provide a catheter assembly for providing embolic protection during an interventional procedure. The catheter includes an outer catheter, an inner catheter, a compliant balloon, and a mesh net. The outer catheter extends from a proximal end to a distal end and has an interior lumen. The inner catheter extends from a proximal end to a distal end of the catheter assembly through the interior lumen of the outer catheter. The compliant balloon is coupled to an exterior surface of the inner catheter at the distal end of the inner catheter. The collapsible mesh is coupled to the exterior surface of the inner catheter proximal to the compliant balloon. The outer catheter is movable between a first position and a second position with respect to the inner catheter, such that in the first position the mesh net is in an expanded configuration and in the second position the mesh net is in a contracted configuration.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 shows a partial view of a catheter assembly according to some embodiments of the disclosure, in which an embolic protection mesh net is shown in an expanded configuration.

FIG. 2 shows a partial view of the catheter assembly of FIG. 2 with the mesh net in a contracted configuration.

FIG. 3 shows an example configuration of an embolic protection mesh net integrated on a catheter assembly.

FIGS. 4A and 4B show an example of a closure mechanism used to close the distal opening of an embolic protection mesh net. FIG. 4A shows the mesh net in an open configuration and FIG. 4B shows the mesh net after the closure mechanism is engaged to close the distal opening of the mesh net.

FIG. 5 shows a schematic view of a catheter assembly having an integrated embolic protection mesh net being deployed in an outflow graft of a left ventricular assist device (“LVAD”).

FIG. 6 is a configuration of a catheter assembly including an embolic protection mesh coupled to an outer catheter that is sized to provide a fluid path between the outer catheter and an inner catheter for the extraction of embolic debris.

FIG. 7 is a configuration of a catheter assembly including an embolic protection mesh that is shaped to form a funnel that when withdrawn causes the mesh net to encompass and secure any embolic debris captured by the mesh net.

FIG. 8 is a configuration of a catheter assembly in which a compliant balloon is shaped on its distal end to form a funnel that directs embolic debris towards the lumen of a catheter for removal.

FIG. 9 is a configuration of a catheter assembly including a mesh braid that can be advanced through an obstruction (e.g., plaque buildup), expanded, and then retracted to dislodge, breakup, or otherwise remove the obstruction.

FIG. 10 is a configuration of a catheter assembly including an embolic protection mesh net, a compliant balloon shaped on its distal end to form a funnel that directs embolic debris towards a lumen for removal, and a mesh braid that can be advanced through an obstruction (e.g., plaque buildup), expanded, and then retracted to dislodge, breakup, or otherwise remove the obstruction.

FIG. 11 shows a delivery sheath for delivering and deploying the embolic protection, debris capture, and plaque removal components of the catheter assembly of FIG. 10 to a location within a patient's vasculature or a medical device, such as an LVAD.

FIG. 12 shows a basket sheath for deploying an embolic mesh net as part of the catheter assembly shown in FIG. 10.

FIG. 13 shows a dual lumen catheter and balloon for deploying the funnel-shaped compliant balloon as part of the catheter assembly shown in FIG. 10.

FIG. 14 shows the dual lumen structure of the dual lumen catheter shown in FIG. 13.

### DETAILED DESCRIPTION

Described herein is an embolic protection device that can be adapted for use with different catheter systems for a number of different clinical applications. In general, the embolic protection device includes a mesh net that is coupled to the exterior surface of a first catheter, which may be a balloon catheter or other catheter used in an interventional procedure. A second catheter is translatable over the first catheter. When the second catheter is retracted proximally along the first catheter, the mesh net is allowed to expand to its opened state where it makes contact with the interior surface of the lumen into which the catheter is deployed. The second catheter can then be translated distally along the first catheter to slide over the mesh net, thereby collapsing the mesh net into its closed state. The catheter assembly is deployed into the lumen in the patient with the mesh net in this closed state, then actuated to retract the second catheter to expand the mesh net into its open state when in use.

As one non-limiting example, the embolic protection device can improve pump thrombosis treatment in patients with mechanical circulatory support (“MCS”) device. A left ventricular assist device (“LVAD”) is a common type of MCS device used in patients with end-stage heart failure. LVADs are surgically implanted mechanical pumps designed to assist the left ventricle of the heart in pumping blood to the rest of the body. LVADs include an inflow conduit extending from the left ventricle to a mechanical LVAD pump, and an outflow graft extending from the LVAD pump to the aorta. Thus, LVADs help pump blood from the left ventricle to the aorta and out to the rest of the body. Over the course of LVAD operation, thrombi can form in the interior of the LVAD pump and on the pump impellor. This phenomenon is known as pump thrombosis.

Approximately ten percent of patients that undergo implantation of an axial flow LVAD develop pump thrombosis with an incidence that is increasing over time. Current lytic therapies for pump thrombosis can result in embolus formation in the blood stream. If the risk of embolus migration were reduced, lytic therapies for pump thrombosis would be a significantly safer option. Embodiments of the disclosed embolic protection system mitigate these risks and expand the population of heart failure patients that could benefit from MCS therapy.

In some embodiments, the disclosed embolic protection system includes a catheter assembly having an outer catheter, an inner catheter, a compliant balloon coupled to the inner catheter, and a mesh net coupled to the exterior surface of the inner catheter at a location that is proximal to the compliant balloon. When the catheter assembly is deployed into the outflow graft of an LVAD, the mesh net can be used to retrieve the thrombolytic debris generated during pump thrombosis therapy. For instance, in pump thrombosis therapy, lytic agents can be administered while the catheter assembly is deployed. After lytic agents are administered upstream through the inflow conduit the mesh net can be deployed to capture thrombolytic debris that is removed from the LVAD in the outflow graft downstream.

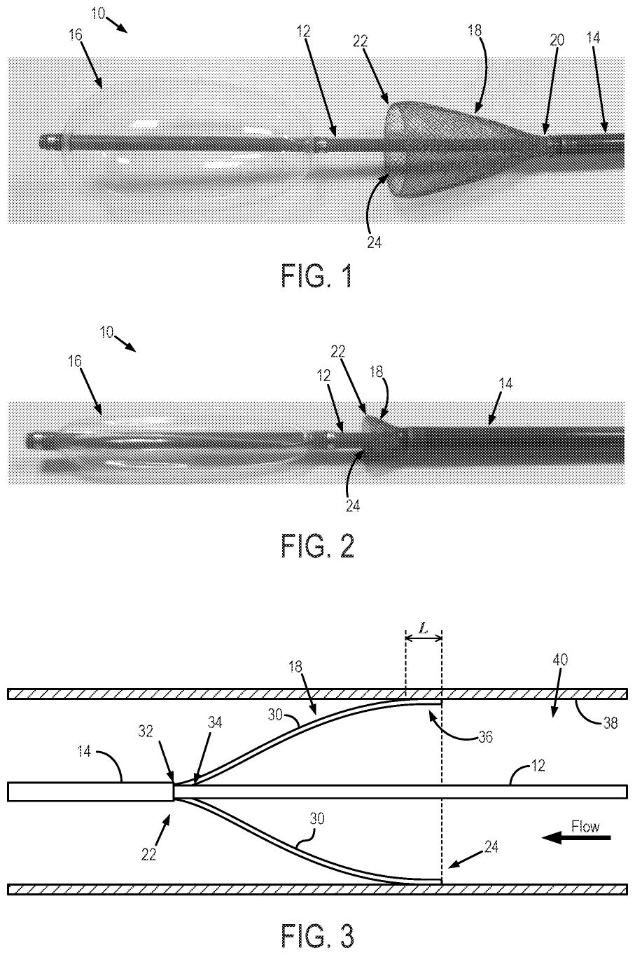

Referring first to FIGS. 1 and 2, a catheter assembly 10 according to some embodiments is illustrated. The catheter assembly 10 can be sized and shaped to be deployed within an outflow graft of an LVAD (e.g., about 9 F, or sometimes larger). In other configurations, the catheter assembly 10 can be sized and shaped for other uses, such as to be deployed in the aorta for the placement of a replacement valve in a transcatheter aortic valve replacement (“TAVR”) procedure, or to be deployed in other blood vessels or bodily lumens.

In general, the catheter assembly 10 includes an inner catheter 12 and a larger outer catheter 14 that can be slid over the inner catheter 12. In some configurations, a compliant balloon 16 is arranged at, or otherwise coupled to, the distal end of the inner catheter 12. This compliant balloon 16 may be used for blocking the outflow graft of an LVAD during a lytic pump thrombosis procedure, for deploying a TAVR device, or so on. In some other configurations, a compliant balloon 16 may not be present on the inner catheter 12. A mesh net 18 that functions as an embolic protection net is arranged at, or otherwise coupled to, the distal end of inner catheter 12. When the inner catheter 12 includes a compliant balloon 16, the mesh net 18 is located at a position that is proximal to the compliant balloon 16.

As a non-limiting example, the compliant balloon 16 is coupled to the exterior surface of the inner catheter 12 at the distal end of the inner catheter 12. The compliant balloon 16 is fluidly coupled to the inner catheter 12 such that the compliant balloon 16 is in fluid communication with a source of fluid (not shown) coupled to the proximal end of the inner catheter 12. Accordingly, the source of fluid can selectively provide a fluid (e.g., liquid or gas) through a lumen in the inner catheter 12 and into the compliant balloon 16 in order to inflate, fill, or otherwise expand the compliant balloon 16 outward from the exterior surface of the inner catheter 12, as shown in FIG. 1. When the compliant balloon 16 is inflated, the pressure of the exterior surface of the compliant balloon 16 anchors the catheter assembly 10 in place within the outflow graft of an LVAD, a blood vessel, or other bodily lumen into which the catheter assembly 10 has been deployed. To deflate the compliant balloon 16, the provided fluid is removed (e.g., via the lumen of the inner catheter 12). The compliant balloon 16 is shown in a partially deflated state in FIG. 2.

The mesh net 18 is coupled to the exterior surface of the inner catheter 12. The outer catheter 14 is sized such that it can be slid over the inner catheter 12 and the mesh net 18. Sliding the outer catheter 14 over the mesh net 18 causes the mesh net 18 to collapse into a closed state. When the outer catheter 14 is slid proximally the mesh net 18 is exposed and expands into its open state.

As noted above, the mesh net 18 is located proximal to the compliant balloon 16 (when present) at a predetermined distance from the compliant balloon 16. The distance between the mesh net 18 and the compliant balloon 16 can be between 0.5 and 1.5 inches, or another distance that facilitates the capture of thrombolytic debris by the mesh net 18 when in its deployed state. In some configurations, there may be no separation distance between the proximal end of the compliant balloon 16 and the distal end of the mesh net 18.

The total diameter of the catheter assembly 10 in its undeployed state (i.e., when the compliant balloon 16 is deflated and the mesh net 18 is in its closed state) is sized to be inserted into the outflow graft of an LVAD, a blood vessel, or other bodily lumen. For example, the outer diameter of the catheter assembly 10 in this undeployed state can be on the order of 9 F.

In general, the holes in the mesh net 18 are sized to prevent the flow of thrombolytic debris through the mesh net 18, while allowing the flow of blood through the mesh net 18. The mesh net 18 is composed of a material such that the mesh net 18 is flexible enough to be collapsed into its closed state by sliding the outer catheter 14 over the mesh net 18. Further, the mesh net 18 is composed of a material such that the mesh net 18 will expand into a defined shape when the outer catheter 14 is retracted. In general, the mesh net 18 is composed of one or more shape-memory materials or smart materials. As one non-limiting example, the mesh net 18 can be composed of a shape-memory alloy, such as nitinol and the like. As another non-limiting example, the mesh net 18 can be composed of a shape-memory polymer, such as polyurethane or block copolymers thereof, and the like.

In its expanded form (i.e., its open state), the mesh net 18 can be shaped such that its diameter increases from its proximal end 20 to its distal end 22. As one example, the mesh net 18 can have a generally conical shape. In its open state, the outer diameter of the mesh net 18 at its distal end 22 is sized so as to make contact with the inner diameter of the outflow graft of an LVAD, a blood vessel, or other bodily lumen into which the catheter assembly 10 is deployed. In this way, when the mesh net 18 is expanded it presses against the inner wall of the lumen into which the catheter assembly 10 has been deployed, thereby holding the mesh net 18 in place and preventing any thrombolytic debris to pass beyond the mesh net 18. In this open state, thrombolytic debris is carried by blood flow into the opening 24 of the mesh net 18 where it is then captured and retained by the mesh net 18.

Because the mesh net 18 is compliant, it can advantageously be sized such that the outer diameter of the mesh net 18 at its greatest radial extent (e.g., at the distal end 22 of the mesh net 18) is greater than the inner diameter of the lumen into which the catheter assembly 10 is deployed. In this way, the mesh net 18 will have a more secure adherence to the inner wall of the lumen by providing an expansive force (e.g., radial tension) against the inner wall of the lumen. Thus, the mesh net 18 can be oversized relative to the lumen into which the catheter assembly 10 will be deployed.

As shown in FIG. 3, the mesh net 18 can be composed of a single layer 30 (i.e., a single layer of mesh) that is coupled to the inner catheter 12 at a first attachment point 32 from which the single layer 30 extends distally before folding over at the distal end 22 of the mesh net 18 to then extend proximally back to a second attachment point 34 where the mesh net 18 is coupled again to the inner catheter 12. In some configurations, a radial support loop may be present between the folded over layer at the distal end 22 of the mesh net 18.

As also shown in FIG. 3, the mesh net 18 can be shaped such that in its opened state the mesh net 18 can have an extended portion 36 at its distal end 22, at which the outer diameter of the mesh net 18 varies little or not at all. In this way, the extended portion 36 of the mesh net 18 will generally be parallel with the inner wall 38 of the lumen 40 into which the catheter assembly 10 is deployed. This extended portion 36 provides an increased surface area for the mesh net 18 to make contact with and anchor to the inner wall 38 of the lumen 40, thereby more securely anchoring the catheter assembly 10. In addition, the extended portion 36 facilitates the capture of thrombolytic debris by the mesh net 18 by having increased contact with the inner wall 38 of the lumen 40. The extended portion 36 can have a length, L, which in some examples can be between 1-5 cm.

This configuration of the mesh net 18 can have particular advantage when deploying the catheter assembly 10 in the aorta, where apposition to the aorta can be challenging. The embolic protection device described in the present disclosure is able to provide sufficient radial tension on the aorta wall without causing damaging to the patient. When used in procedures such as TAVR placement, the embolic protection provided by the catheter assembly 10 can remove the need for embolic nets on the aortic arch.

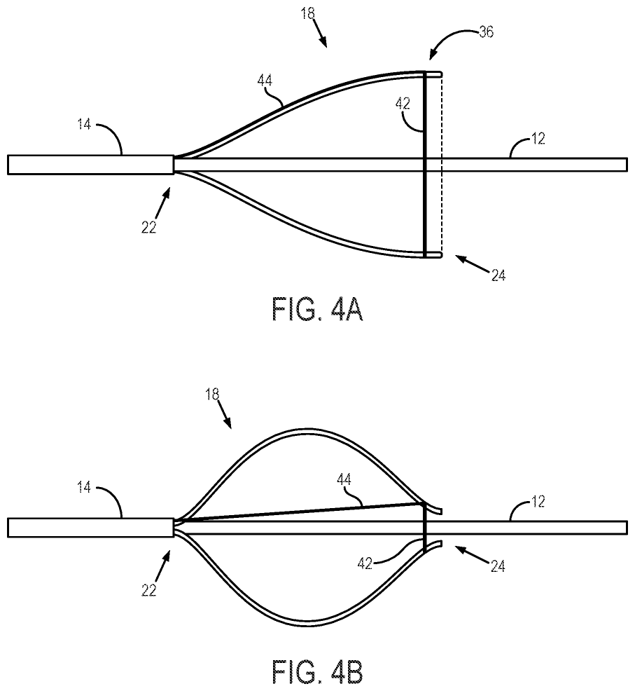

In some configurations, such as those shown in FIGS. 4A and 4B, the mesh net 18 can include a closure mechanism 42 at its distal end 22. For example, a wire or string can be arranged at the distal end 22 of the mesh net 18 such that actuation of the wire or string causes the wire or string to cinch. In such instances, a wire 44 (which may also be a string, cable, or the like) can be coupled to the closure mechanism 42 such that actuation of the wire 44 will result in the closure mechanism 42 to close the opening 24 of the mesh net 18. For instance, as the wire or sting of the closure mechanism 42 is cinched, the outer diameter of the mesh net 18 at its distal end 22 will decrease to close the opening 24 of the mesh net 18, thereby securely capturing any thrombolytic debris contained in the mesh net 18. In these instances, when the outer catheter 14 is slid over the mesh net 18 to collapse the mesh net 18 into its closed state, the thrombolytic debris is prevented from being expelled from the mesh net 18 through the opening 24.

As one example, the closure mechanism 42 can include a wire or string that extends around the circumference of the mesh net 18 proximate the distal end 22 of the mesh net 18, as shown in FIG. 4A. By actuating the wire 44 to engage the closure mechanism 42, the opening 24 of the mesh net 18 can be kept closed during and after contraction and/or closure of the mesh net 18, as shown in FIG. 4B.

As noted above, expansion and contraction of the mesh net 18 is controlled by the movement of the outer catheter 14. The outer catheter 14 is can be translated (e.g., slid) along the inner catheter 12 by the clinician. Because the mesh net 18 is coupled to the exterior surface of the inner catheter 12, the outer catheter 14 can be slid at least partially over mesh net 18. When the outer catheter 14 slides distally over the mesh net 18, the mesh net 18 is contracted against the exterior surface of the inner catheter 12, as shown in FIG. 2. As the outer catheter 14 is retracted proximally, the mesh net 18 expands into its open state, as shown in FIG. 1. Thus, the clinician can control the expansion and contraction of the mesh net 18 by sliding the outer catheter 14.

When the catheter assembly 10 is deployed into the outflow graft of the LVAD, a blood vessel, or other bodily lumen, the outer catheter 14 is positioned over the mesh net 18 such that the mesh net 18 is contracted to a size for the catheter assembly 10 to be deployable within the patient. Once the catheter assembly 10 is in place and the mesh net 18 is ready to be deployed for embolic protection, the outer catheter 14 is retracted proximally along the inner catheter 12 to allow the mesh net 18 to expand. When the mesh net 18 is fully expanded it makes contact with the inner wall of the lumen in which the catheter assembly 10 has been deployed. After the desired procedure (e.g., pump thrombosis therapy, TAVR placement) has been performed, the outer catheter 14 is moved distally to contract the mesh net 18 and capture the thrombolytic debris for removal from the patient. When the mesh net 18 includes a closure mechanism 42, such as those described above, the distal end 22 of the mesh net 18 is closed before sliding the outer catheter 14 over the inner catheter 12 to collapse the mesh net 18 into its closed state.

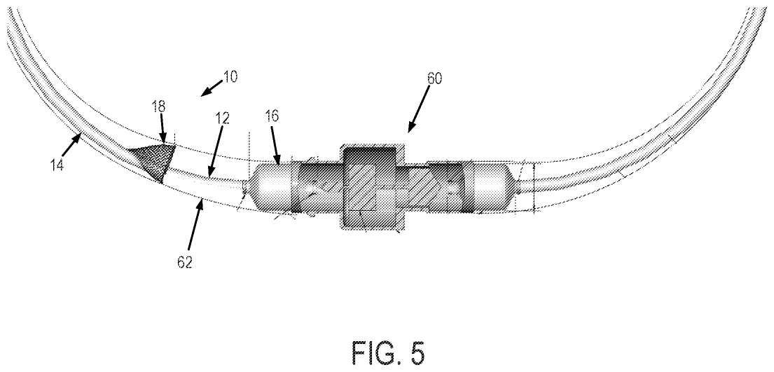

FIG. 5 illustrates an example of a catheter assembly 10 according to some embodiments of the present disclosure in use for pump thrombosis treatment of an LVAD. Once the catheter assembly 10 is deployed into the outflow graft 62 of the patient's LVAD pump 60, the compliant balloon 16 is expanded to anchor the catheter assembly 10 in place. In some forms, the catheter assembly 10 can be held in place by a structure on the distal tip of the inner catheter 12 that directly couples the catheter assembly 10 to the LVAD pump 60. By directly coupling the catheter assembly 10 to the LVAD pump 60, the exact location of the mesh net 18 with respect to the LVAD pump 60 can be ensured. Next, the outer catheter 14 is moved proximally to expand the mesh net 18 against the interior surface of the outflow graft 62. A lytic agent, such as heparin, can be administered and the speed of the LVAD pump motor can then be increased to improve removal of thrombi that have formed inside of the LVAD pump 60. Other lytic agents can also be administered, such as a tissue plasminogen activator to improve removal of thrombi. During the pump thrombosis treatment, the mesh net 18 will catch embolic debris. The mesh net 18 is particularly useful for catching debris after the compliant balloon 16 is deflated and full flow is allowed to pass through the outflow graft 62. When the procedure is complete, the outer catheter 14 is slid distally over the mesh net 18 in order to collapse the mesh net 18 back into its closed state. When the mesh net 18 includes a closure mechanism 42, the opening 24 of the mesh net 18 can be closed using the closure mechanism 42 before sliding the outer catheter 14 over the mesh net 18. In this closed state, the catheter assembly 10 can then be removed along with any thrombolytic debris caught in the mesh net 18.

Referring now to FIG., an alternative configuration of a catheter assembly 10 according to some embodiments of the present disclosure is shown. In this configuration, the mesh net 18 is coupled to an outer catheter 14 that is dimensioned such that there is sufficient space between the inner catheter 12 and the outer catheter 14 to define a passage 70 through which embolic debris can be extracted. In this configuration, the mesh net 18 is coupled to the outer catheter 14 rather than the inner catheter 12. The mesh net 18 can be composed of a silicone coated braid. The opening 24 of the mesh net 18 can be sized and shaped in its deployed state to allow particles to flow into the mesh net 18 and into the passage 70 formed between the inner catheter 12 and the outer catheter 14.

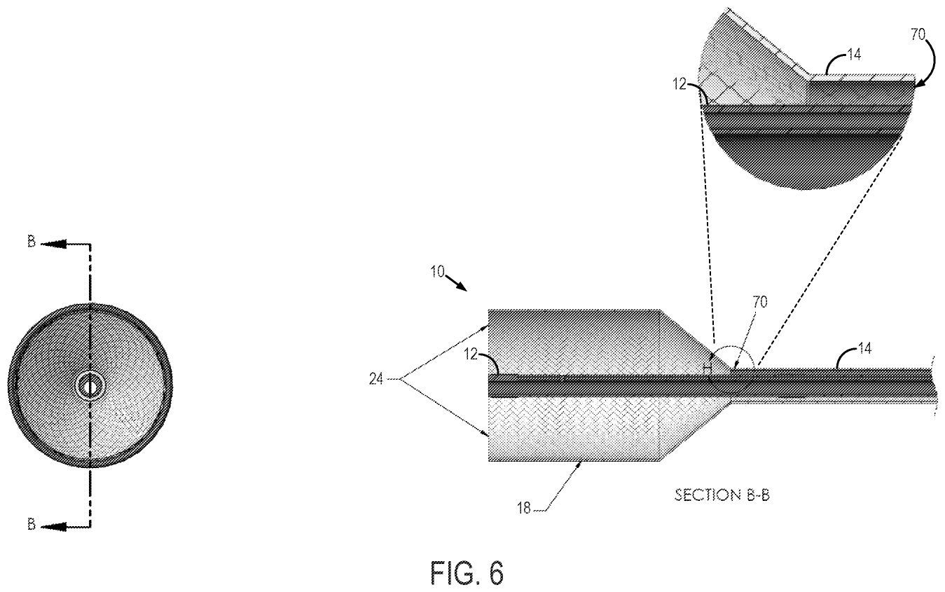

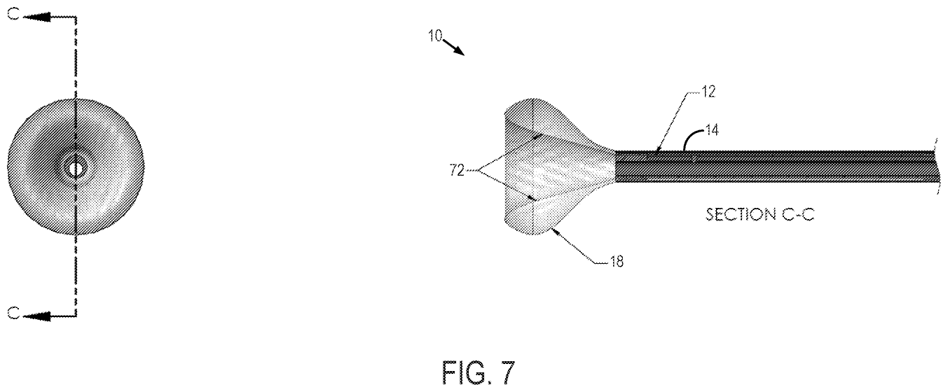

Referring now to FIG. 7, another alternative configuration of a catheter assembly 10 according to some embodiments of the present disclosure is shown. In this configuration, the mesh net 18 is shaped such that in its deployed state it forms a funnel that can trap embolic debris as it is withdrawn. For instance, as the mesh net 18 is withdrawn an outer overlap region 72 of the mesh net 18 will encompass the embolic debris contained within the opening 24 of the mesh net 18. When the mesh net 18 is full withdrawn, the embolic debris is safely removed into the interior of the inner catheter 12. Like the configuration shown in FIG. 6, and other configurations described in the present disclosure, the mesh net 18 may be composed of a silicone coated braid.

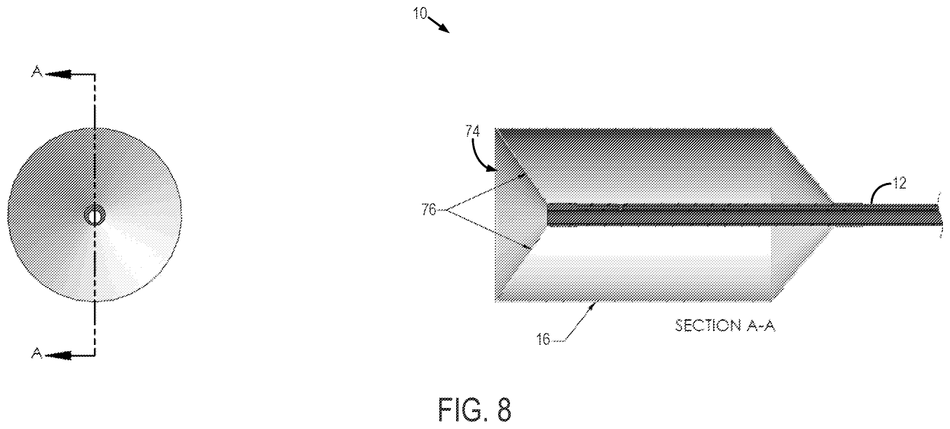

Referring now to FIG. 8, still another alternative configuration of a catheter assembly 10 according to some embodiments of the present disclosure is shown. In this configuration, the outer catheter 14 and mesh net 18 are not used. Instead, the compliant balloon 16 is shaped to enable capture of embolic debris. For example, as shown the compliant balloon includes a funnel shape at its distal end, such that embolic debris will be passed into the opening 74 of the funnel 76 and directed toward the inner catheter 12 for removal. As one example, the compliant balloon 16 may be composed of nylon or another suitable material.

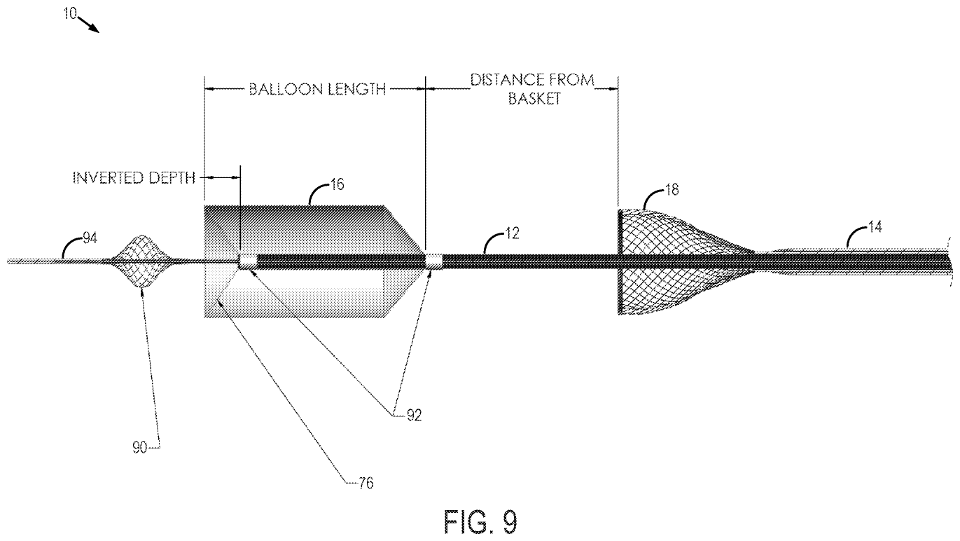

In still other configurations, the catheter assembly 10 can include both a compliant balloon 16 as configured in FIG. 8 and the outer catheter 14 with mesh net 18. An example of such a configuration is shown in FIG. 9. The compliant balloon 16 is located distal to the mesh net 18.

Referring now to FIG. 9, in some embodiments an optional mesh braid 90 can be arranged on an additional internal catheter 94 that can be advanced through the inner catheter 12 distal to the mesh net 18 and compliant balloon 16. This additional mesh braid 90 can be actuated to expand into an expanded state, in which the mesh braid is capable of removing plaque and other debris from the lumen into which the catheter assembly 10 has been provided. For instance, if the flow of fluid remains compromised in the lumen during the evacuation process (e.g., due to plaque buildup or other obstructions), the mesh braid 90 can be advanced out the distal end of the catheter assembly 10 as shown in FIG. 9 and past the obstruction. The mesh braid 90 can then be expanded and retracted in order to dislodge or otherwise breakup the plaque or other obstruction. The evacuation process can then continue.

As shown in FIG. 9, marker bands 92 can also be coupled to the inner catheter 12, such that the position of the catheter assembly 10 can be tracked using a suitable medical imaging modality. For instance, the marker bands 92 can be composed of a radiopaque material such that the marker bands 92 can be imaged with x-ray imaging, such as x-ray fluoroscopy imaging. Similar marker bands 92 can also be coupled to the outer catheter 14 or other portions of the catheter assembly 10.

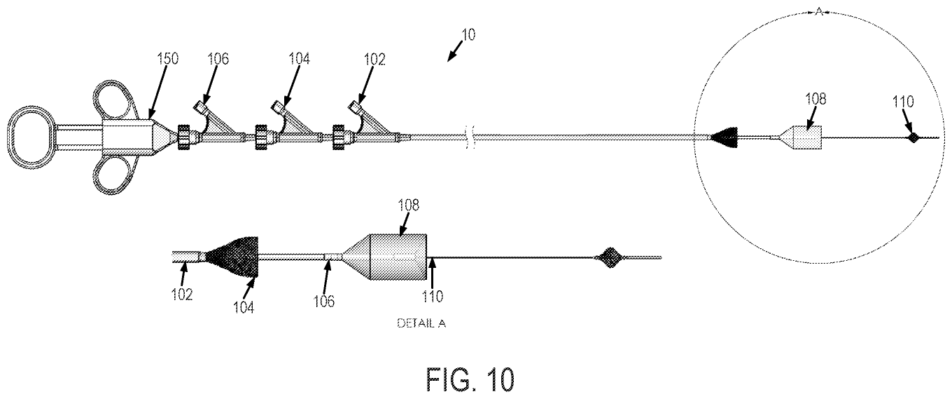

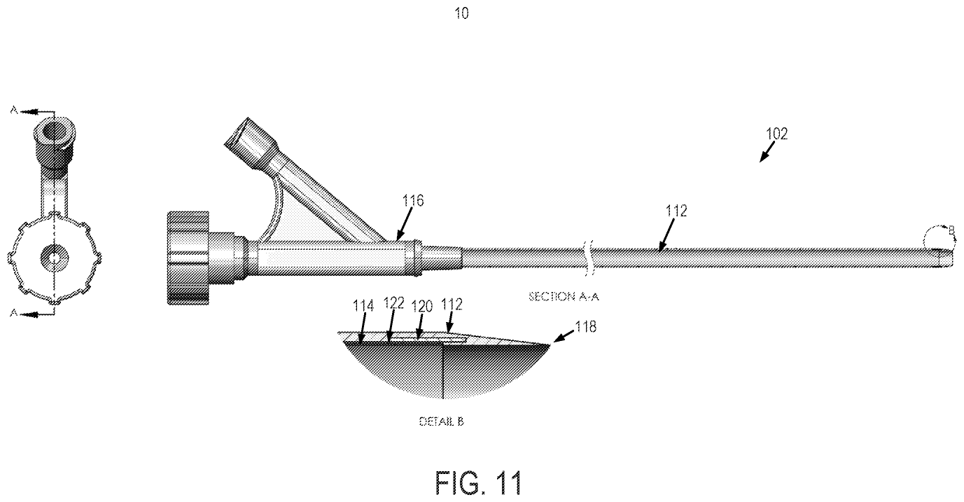

FIG. 10 shows an example catheter assembly 10 for providing embolic protection during an interventional procedure using some of the example components described in the present disclosure. In this example, the catheter assembly 10 generally includes four components: a delivery sheath 102, a basket sheath 104, a dual lumen catheter 106 with a balloon 108, and a plaque remover 110.

As shown in FIG. 11, the delivery sheath 102 can generally include a lumen 112 that extends from a proximal end to a distal end. As a non-limiting example, the interior of the lumen 112 may be coated or composed of PTFE or other similar materials, or may include an inner liner 114 composed of PTFE or other similar materials. At its proximal end, the lumen 112 is coupled to a hemostasis valve 116. A tip 118 can be formed at the distal end of the lumen 112, and one or more marker bands 120 can be coupled to, or otherwise arranged at, the tip 118 to facilitate imaging and localization of the delivery sheath 102. In some implementations, a braid 122, such as a stainless-steel braid, may be disposed within the inner diameter of the lumen 112 to provide structural support for the lumen 112. For instance, the stainless-steel braid 122 can be arranged between the lumen 112 and the inner liner 114. In use, the delivery sheath 102 facilitates the delivery of tools and catheters, such as the compliant balloon 16 and mesh net 18 described above, to a location within a patient's vasculature and/or to a location within an LVAD device. In some implementations, the delivery sheath 102 can be used as the outer catheter 14 of the catheter assembly 10 as described above.

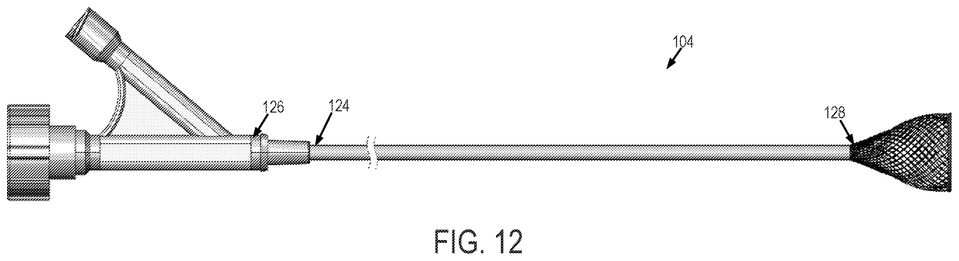

As shown in FIG. 12, the basket sheath 104 can generally include a lumen 124 that extends from a proximal end to a distal end. At its proximal end, the lumen 124 can be coupled to a hemostasis valve 126. A basket 128 is coupled to, or otherwise arranged at, the distal end of the lumen 124. The basket 128 may be, for example, a silicone coated basket. In use, the basket sheath 104 can provide embolic protection during an interventional procedure (e.g., by capturing thrombolytic debris). For instance, the basket sheath 104 can be used as the inner catheter 12 and mesh net 18 of the catheter assembly described above.

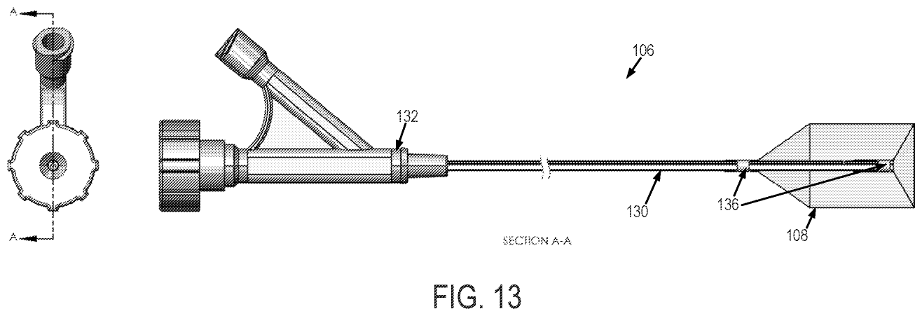

As shown in FIG. 13, the dual lumen catheter 106 with the balloon 108 includes a dual lumen extrusion 130 that extends from a proximal end to a distal end. At its proximal end, the dual lumen extrusion 130 is coupled to a hemostasis valve 132. A balloon 108 is coupled to, or otherwise arranged at, the distal end of the dual lumen extrusion 130, and one or more marker bands 136 can also be coupled to, or otherwise arranged at, the distal end of the dual lumen extrusion 130. As described above, the marker band(s) 136 can provide for tracking of the catheter assembly 10 using a suitable medical imaging modality. The balloon 108 can be shaped to enable capture of embolic debris. For example, as shown the compliant balloon includes a funnel shape at its distal end, such that embolic debris will be passed into the opening of the funnel and directed toward the dual lumen extrusion 130 for removal. Similar to the embodiments described above, the balloon 108 may be composed of nylon or another suitable material.

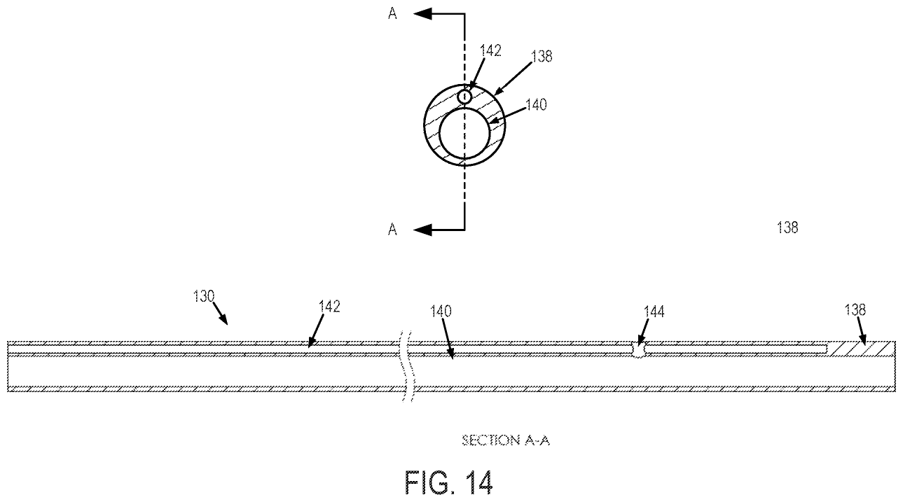

An example of the dual lumen extrusion 130 is illustrated in FIG. 14. In general, the dual lumen extrusion 130 includes a tubular structure 138 having a first lumen 140 and a second lumen 142 extending therethrough. The first lumen 140 generally has a larger inner diameter than the second lumen 142 and is thus configured for the transport and removal of embolic debris and the like. The second lumen 142 has a smaller inner diameter than the first lumen 140 and is generally configured to deliver fluid to the inner volume of the balloon 108, such that when fluid is provided to the inner volume of the balloon 108 the balloon 108 is made to expand. As such, the first lumen 140 may extend fully from the proximal end to the distal end of the tubular structure 138, whereas the second lumen 142 may extend from the proximal end of the tubular structure 138 only partially towards the distal end of the tubular structure 138. An aperture 144 extends from the outer surface of the tubular structure 138 to the inner diameter of the second lumen 142 to provide a fluid coupling between the second lumen 142 and the inner volume of the balloon 108.

As shown in FIG. 10, the plaque remover 110 may include a mesh braid, such as mesh braid 90 described above, or the like, that can be deployed for breaking up and otherwise removing plaque. The embolic debris created by breaking up plaque with the plaque remover 110 can then be safely captured, such as by the balloon 108 and/or basket 128. As also shown in FIG. 10, the plaque remover 110 can be actuated at the proximal end of the catheter assembly 10 using a handle 150, or the like.

Thus, an improved system for treating pump thrombosis is provided that offers a less invasive procedure and decreases the chance of adverse effects caused by emboli that may be formed from thrombolytic debris during treatment.

The present invention has been described in terms of one or more preferred embodiments, and it should be appreciated that many equivalents, alternatives, variations, and modifications, aside from those expressly stated, are possible and within the scope of the invention.

1. A catheter assembly for providing embolic protection during an interventional procedure, comprising:
an outer catheter extending from a proximal end to a distal end and having an interior lumen;
an inner catheter having an exterior surface, the inner catheter extending from a proximal end to a distal end through the interior lumen of the outer catheter;
a compliant balloon coupled to the exterior surface of the inner catheter at the distal end of the inner catheter;
a mesh net coupled to the exterior surface of the inner catheter proximal to the compliant balloon; and
wherein the outer catheter is movable between a first position and a second position with respect to the inner catheter, such that in the first position the mesh net is in an expanded configuration and in the second position the mesh net is in a contracted configuration.

2. The catheter assembly of claim 1, wherein the mesh net has a diameter that increases from a proximal end to a distal end as the mesh net extends distally along a longitudinal axis of the catheter assembly.

3. The catheter assembly of claim 2, wherein the mesh net includes an extended portion at its distal end over which the diameter of the mesh net is substantially constant.

4. The catheter assembly of claim 2, wherein the mesh net comprises a single layer coupled to and extending distally from the inner catheter at a first attachment point, folded over at the distal end of the mesh net, and coupled to the inner catheter at a second attachment point that is distal to and adjacent the first attachment point.

5. The catheter assembly of claim 1, wherein the mesh net is composed of a shape-memory material.

6. The catheter assembly of claim 5, wherein the shape-memory material is a shape-memory alloy.

7. The catheter assembly of claim 6, wherein the shape-memory alloy is nitinol.

8. The catheter assembly of claim 5, wherein the shape-memory material is a shape-memory polymer.

9. The catheter assembly of claim 1, wherein the mesh net is contracted into a closed state when the outer catheter is moved distally over the mesh net from the first position to the second position.

10. The catheter assembly of claim 1, wherein the mesh net is spaced apart from the complaint balloon by a separation distance between 0 and 1 inch.

11. The catheter assembly of claim 1, further comprising a closure mechanism arranged at the distal end of the mesh net and operable to reduce the diameter of the mesh net at the distal end of the mesh net.

12. The catheter assembly of claim 11, wherein the closure mechanism comprises one of a string or wire extending around a circumference of the mesh net at the distal end of the mesh net.

13. The catheter assembly of claim 11, wherein the closure mechanism is operated to close the mesh net by actuating one of a wire or a string coupled to the closure mechanism.

14. A catheter assembly for providing embolic protection during an interventional procedure, comprising:
an outer catheter extending from a proximal end to a distal end and having an interior lumen;
an inner catheter having an exterior surface, the inner catheter extending from a proximal end to a distal end through the interior lumen of the outer catheter;
a mesh net coupled to the distal end of the outer catheter; and
wherein an inner diameter of the outer catheter and an outer diameter of the inner catheter are dimensioned such that when the inner catheter is position within the interior lumen of the outer catheter a passage is formed therebetween to define a fluid path for extracting embolic debris caught by the mesh net.

15. A catheter assembly for providing embolic protection during an interventional procedure, comprising:
a catheter having an exterior surface, the catheter extending from a proximal end to a distal end;
a compliant balloon having a first end and a second end, wherein the first end of the compliant balloon is coupled to the exterior surface of the catheter at the distal end of the catheter and the second end of the compliant balloon is coupled to the exterior surface of the catheter proximal to the distal end of the catheter; and
wherein the compliant balloon is shaped at its first end to form a funnel comprising an interior surface extending distally from a narrow end having a narrow outlet opening that is coupled to a lumen of the catheter to a wide end having a wide inlet opening.

16. A catheter assembly for providing embolic protection during an interventional procedure, comprising:
an outer catheter extending from a proximal end to a distal end and having an interior lumen;
an inner catheter extending from a proximal end to a distal end through the interior lumen of the outer catheter and having an interior lumen;
a mesh net coupled to the distal end of the inner catheter;
a dual lumen catheter having an exterior surface and extending from a proximal end to a distal end through the interior lumen of the inner catheter, the dual lumen catheter having a first interior lumen and a second interior lumen;
a compliant balloon having a first end and a second end, wherein the first end of the compliant balloon is coupled to the exterior surface of the dual lumen catheter at the distal end of the dual lumen catheter and the second end of the compliant balloon is coupled to the exterior surface of the dual lumen catheter proximal to the distal end of the dual lumen catheter;
wherein the compliant balloon is shaped at its first end to form a funnel comprising an interior surface extending distally from a narrow end having a narrow outlet opening that is coupled to the first interior lumen of the dual lumen catheter to a wide end having a wide inlet opening;
wherein the second interior lumen of the dual lumen catheter is fluidically coupled to an inner volume of the compliant balloon providing fluid through the second interior lumen causes the compliant balloon to expand into an expanded state; and
wherein the outer catheter is movable between a first position and a second position with respect to the inner catheter, such that in the first position the mesh net is in an expanded configuration and in the second position the mesh net is in a contracted configuration.
